(19) United States
(12) Patent Application Publication
(10) Pub. No.: US 2012/0253232 A1
(43) Pub. Date: Oct. 4, 2012

(54) METHOD FOR CREATING COLOR SCALE FOR DETERMINATION OF MASTICATORY PERFORMANCE

(76) Inventors: Shunsuke Minakuchi, Tokyo (JP); Tatsuro Uchida, Tokyo (JP); Manabu Kanazaka, Tokyo (JP); Makoto Sato, Saitama-shi (JP); Yasutaka Hiraoka, Saitama-shi (JP); Ryota Sasaki, Tokyo (JP); Daigo Sugita, Tokyo (JP)

(21) Appl. No.: 13/498,140

(22) PCT Filed: Sep. 30, 2010
(86) PCT No.: PCT/JP2010/005890

§ 371 Date: Jun. 4, 2012

(30) Foreign Application Priority Data
Sep. 30, 2009 (JP) 2009-227154

(51) Int. Cl. A61B 5/11

(52) U.S. Cl. 600/590

(57) ABSTRACT

A method for creating a color scale which can be used as a more objective determination criteria in order to analyze the color change characteristics of a gum with the progression of mastication of individuals with healthy teeth and jaw and to use for evaluation of masticatory performance by a color-changeable chewing gum is provided. A method for creating a color scale in which the number of times of mastication by individuals with healthy teeth and jaw and the color difference of the color developed by the gum after mastication are determined by having a plurality of individuals with healthy teeth and jaw masticate a color-changeable chewing gum to determine a regression equation of the color developed by the gum after mastication and the color difference of gums before and after mastication and further determining a regression equation of the number of times of mastication and the color difference of gums before and after mastication.

### TECHNICAL FIELD

The present invention relates a method for creating a color scale for determination of masticatory performance.

### BACKGROUND ART

Various methods for directly determining masticatory performance have been developed and reported. Methods for directly determining masticatory performance are roughly classified into a subjective method in which a subject is questioned about an ingestible foodstuff and a method in which the state of actually masticated sample is objectively evaluated. Among these, although the latter method not requiring subjective judgment by a subject can evaluate masticatory performance quantitatively, since its operation is complex and specific instruments are required, it cannot be readily carried out in the field of dental diagnosis or in a daily life.

In order to solve these problems, the present inventors have developed a color-changeable chewing gum whose color changes with the progression of mastication and which is actually sold as Masticatory Performance Evaluating gum XYLITOL (trade name). The color of the color-changeable chewing gum changes from green to pink with the progression of mastication and masticatory performance can be readily determined by evaluating the color after mastication. A chewing gum has stable properties as a material and uniform products can be produced in large quantities. Further, a chewing gum has various advantages as a sample for measurement, for example, it is taken orally on a daily basis and it is food having a uniform texture.

The present inventors have also tried to measure masticatory performance by the color-changeable chewing gum. For example, Non Patent Literatures 1 and 2 report that there is a relationship in the results between the evaluation method of masticatory performance by the color-changeable chewing gum and the conventional sieving method, and that the degree of color change of the color-changeable chewing gum proceeds with the progression of mastication. In order to evaluate masticatory performance more conveniently, Non Patent Literature 3 discloses the reliability among examiners of the method in which an experimental color scale is created and masticatory performance is compared by comparing the color change of the color-changeable chewing gum with the color scale. That is, when a plurality of examiners keep the score using the color scale created in Non Patent Literature 3 by observing the gum after mastication, the results will become the same score, irrespective of those who keep the score. This result shows that the color of the gum can be evaluated by the color scale with high reproducibility.

However, the color scale used in Non Patent Literature 3 is visually created from the color tone of the masticated gum and it is not created by quantitatively studying the color change. In addition, the color scale is created based on the mastication results by randomly selected dentists. For this reason, in order to create a color scale which enables a more objective and quantitative determination, detailed studies on the color change of a gum during mastication from immediately after the start of mastication when a subject having an average masticatory performance masticates the gum have been demanded.

### CITATION LIST

### Non Patent Literature

NPL 1: Hayakawa et al: “A Simple Method for Evaluating Masticatory Performance Using a Color-Changeable Chewing Gum” The International Journal of Prosthodontics 11(2): pp173-176, 1998.

NPL 2: Hirano et al: “A Study on Measurement of Masticatory Ability Using a Color-changeable Chewing Gum with a New Coloring Reaction” Journal of Prosthodontic Research, Vol. 46, pp103-109, 2002.

NPL 3: Fujinami et al: “Reliability of Evaluation Method of Masticatory Ability by Color-Changeable Chewing Gum Using Color Scale” Journal of Japanese Society for Mastication Science and Health Promotion, Vol. 18, No. 2 pp173-174, 2008.

### SUMMARY OF INVENTION

### Technical Problem

It is an object of the present invention to create a color scale which can be used as a more objective and quantitative determination criteria for evaluating masticatory performance by a color-changeable chewing gum.

### Solution to Problem

In light of the above problems, the present invention relates to a method for creating a color scale for determining a number of times of mastication of an individual with healthy teeth and jaw, using a gum with a color changing depending on mastication, based on a color of the gum after mastication, the method comprising a first step of having a plurality of individuals with healthy teeth and jaw masticate the gum with a color changing depending on mastication a fixed number of times; a second step of determining a regression equation representing a relationship between a coordinate value of a color of the gum after mastication in a specific color space and a color difference in the color space of the gum between before and after mastication in a color system; a third step of determining a regression equation representing a relationship between the color difference and the number of times of mastication; and a fourth step of determining an average number of times of mastication for the color of the gum after mastication from the regression equations obtained in the second and third steps.

### Advantageous Effect of Invention

According to the method of the present invention, a color scale for determining mastication performance more objectively and quantitatively, compared with the conventional method, can be created.

### BRIEF DESCRIPTION OF DRAWINGS

FIG. 1 is a graph showing the relationship between ΔE and L*a*b* in one Example of the present invention.

FIG. 2 is a graph showing the relationship between ΔE and the number of times of mastication in one Example of the present invention.

### EXAMPLES

An unlimited method for creating the color scale created in the present Example will be illustratively described below. In this regard, it should be noted that the present invention is not limited to the following constitution for the subjects or the number of subjects, the number of times of mastication, the method for color measurement, and the like.

The term “mastication performance” as used herein means the ability of crushing and mixing food to blend with saliva. The mastication performance in the present invention can be represented by, determining the degree in which a chewing gum is crushed and mixed to blend with the saliva in masticating a chewing gum for a fixed number of times, counting the number of times of mastication required for individuals with healthy teeth and jaw to crush and mix a chewing gum to blend with saliva to the same degree as above, and comparing the number of times of mastication.

### 1. The Color Change Characteristics of a Gum With Progression of Mastication

In order to investigate the color change characteristics due to the number of times of mastication of individuals with healthy teeth and jaw, Masticatory Performance Evaluating gum XYLITOL is masticated for a predetermined number of times of mastication and the color of the gum after mastication for each of the numbers of times of mastication and the color difference of the gums between before and after mastication was investigated.

The subjects were 61 individuals with healthy teeth and jaw without deficiency except for the third molar (38 males and 23 females with a mean age of 29.2 years old) having no abnormalities of stomatognathic system. Every subject was given Masticatory Performance Evaluating gum XYLITOL and given instructions to masticate a gum well, followed by having the subjects masticate the gum by each predetermined number of times of mastication. The number of times of mastication was set at 20, 40, 60, 80, 120 and 160 and each subject performed the trial once for every mastication for total 6 times. No instruction was given as to which side of the mouth was used to masticate the gum. Each trial was performed after more than two hours had passed after a meal without drinking and eating except for water.

The used chewing gum is the same as those actually sold, which is a gum of a type of substantially stick form (36×20×5 mm, 3.0 g) and contains, as main ingredients, a gum base, citric acid, xylitol, red, yellow and blue pigments and the like. Since the gum is in the state where the pH is low due to the presence of citric acid and synthetic colorants which do not develop color in an acidic region are used as red pigments, the gum before mastication has a yellowish green color due to the presence of yellow and blue pigments. When the gum is blended with the saliva with the progression of mastication, yellow and blue pigments are dissolved out from the gum base. Simultaneously, the pH inside the gum increases due to the citric acid dissolved out into the saliva, and thus the red pigment develops a red color. As a result, the gum turns from yellowish green to red. In addition, as long as the conditions that the color of the gum can be changed due to mastication are satisfied, there is no limitation on the gum ingredients other than the above ingredients or colorant ingredients, and any color-changeable chewing gum may be used. The composition of the Masticatory Performance Evaluating gum XYLITOL used in the present Example is shown in Table 1.

TABLE 1

| | |
|---|---|
| Gum base | 23% |
| Sugar alcohols (xylitol, maltitol) | 75% |
| Perfume and others | 2% |
| Citric acid | 0.1% |
| Food Red No. 3 (in gum) | 0.01% |
| Food Yellow No. 4 (in gum) | 0.01% |
| Food Blue No. 1 (in gum) | 0.001% |

A gum was collected immediately after mastication and pressed into a thickness of 1.5 mm through a polyethylene film using two glass plates. Subsequently, the glass plates were removed and the color of the gum was measured from above the polyethylene film using a color reader (CR-13, manufactured by Konica Minolta Sensing, Inc.). The color measurement was performed at five points in total: one point at the center of the pressed gum and four points apart from the center to the top, bottom, left and right by approximately 3 mm and the average value at five points was used for analysis. A CIE L*a*b* color system was used for the color measurement of the gum.

The CIE L*a*b* color system is one of the uniform color spaces established by CIE (Commission Internationale de l'Eclairage) in 1976. The color system is most frequently used for representing the color of objects in digitization and it is a color space capable of displaying the difference in color perceived by vision from the distance of a point represented by three coordinates L*, a* and b* with high reproducibility. Although the CIE L*a*b* color system is used in the present Example to create a color scale which can visually determine masticatory performance later, any color space may be used as long as the number of times of mastication can be regressed from the measurement values by one-to-one correspondence.

### 2. Measurement Results 1: Relation Between ΔE and L*, a* and b*

The values of each of L*, a* and b* of the colors of the chewing gum before mastication and the chewing gum after mastication in each of subjects, which were measured in the above step, were determined and the color difference ΔE in the chewing gums before and after mastication was calculated.

The color difference ΔE is a value representing variations in color and it is represented by the distance between the coordinate of the color before change and the coordinate of the color after change in a specific color space. In the CIE L*a*b* color system used in the present Example, if the average values of L*, a* and b* after mastication are defined as Lₓ, aₓ and bₓ, respectively, and the average values of L*, a* and b* of the gum before mastication as a control measured in the same manner as above are defined as L₀, a₀ and b₀, ΔE can be determined by the expression: ΔE={(Lₓ−L₀)²+(aₓ−a₀)²+(bₓ−b₀)²}^{1/2}. In addition, in order to create a color scale by a method in the present invention using other color systems, for example, if the measurement variables in other color systems are defined as X, Y and Z, a function f_{(X,Y,Z)}, which can regress the number of times of mastication, may exist. In the present Example, the f(_{X,Y,Z)} is a function of ΔE.

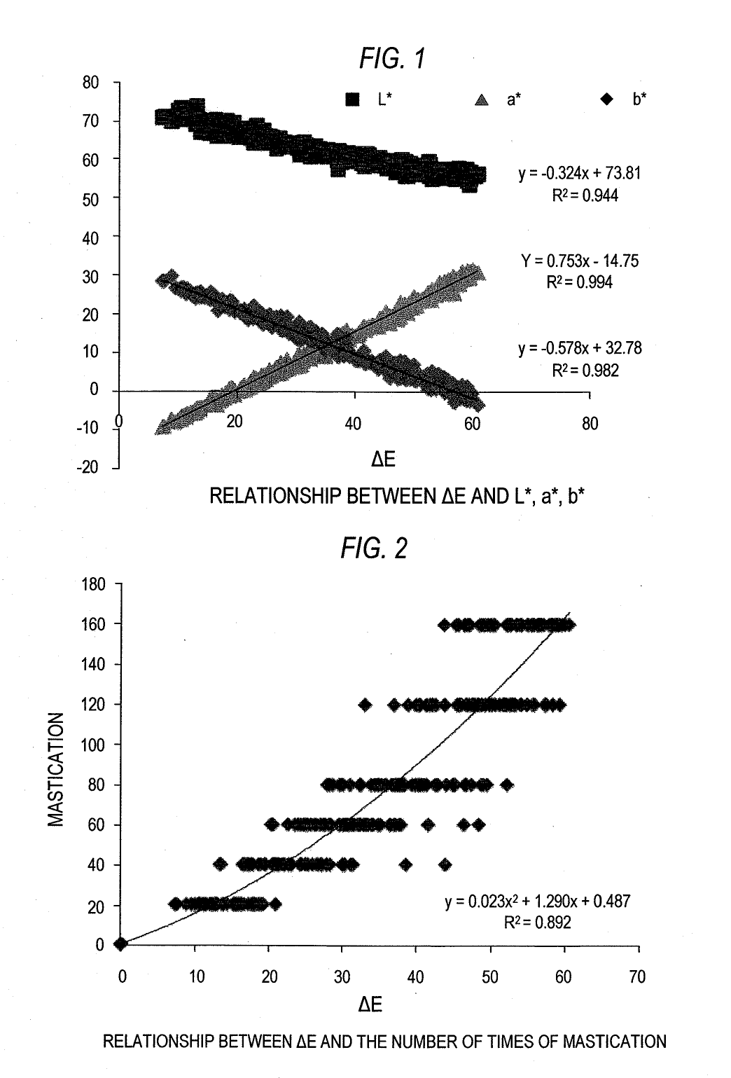

The relationship between the color difference ΔE determined and the values of L*, a* and b* is shown in FIG. 1. It is understood from FIG. 1 that ΔE and the values of L*, a* and b* show strong correlation.

The results of FIG. 1 suggest that the color of the gum changes with the progression of mastication along a specific straight line on the color space. That is, it is considered that by determining a color difference ΔE of the color of the gum before and after mastication and moving on the straight line by the color difference ΔE, a position (the values of L*, a* and b* in the present Example) on the color space corresponding to the color difference ΔE can also be determined.

### 3. Measurement Result 2: Relationship Between ΔE and the Number of Times of Mastication

The relationship between the ΔE thus determined and the number of times of mastication is shown in FIG. 2. It is understood from FIG. 2 that ΔE and the number of times of mastication show strong correlation. It is understood from the results that an average color difference ΔE to the number of times of mastication can be determined by determining a regression equation of number of times of mastication and color difference ΔE.

In the present Example, ΔE and the number of times of mastication can regress to the quadratic curve shown in FIG. 2. The results show that an average number of times of mastication required for the quantity of the color change may be determined for the gum which is changed in color by mastication using the determined quadratic curve.

### 4. Creation of Color Scale

It is understood from the results of the above 2. that the values L*, a* and b* of the color of the chewing gum after mastication has a one-to-one relationship with the color difference ΔE of the color of the chewing gums after mastication. In addition, it is understood from the results of the above item 3 that ΔE and the number of times of mastication are represented by a one-to-one relationship.

That is, a regression equation representing the relationship between ΔE and L*, a* and b* is determined from the results of the above item 2 and a regression equation representing the relationship between each of the number of times of mastication and ΔE is determined from the results of above item 3, thereby enabling determining the typical values of L*, a* and b* of the colors of the gums after mastication to the number of times of mastication in individuals with healthy teeth and jaw. The number of times of mastication, the value of ΔE corresponding to the number and the values of L*, a* and b*, which are thus obtained, are shown in Table 2. Based on the results, a color scale representing the relationship between the number of times of mastication and the colors of the gums after mastication in individuals with healthy teeth and jaw was created.

TABLE 2
Number of times of mastication and data on each color in individuals with healthy teeth and jaw

| | Number of times | | | | | | | | | | | | | | | | |
|---|---|---|---|---|---|---|---|---|---|---|---|---|---|---|---|---|---|
| | 0 | 10 | 20 | 30 | 40 | 50 | 60 | 70 | 80 | 90 | 100 | 110 | 120 | 130 | 140 | 150 | 160 |
| ΔE | 0 | 7 | 12 | 17 | 22 | 26 | 30 | 33 | 37 | 40 | 43 | 46 | 49 | 52 | 54 | 57 | 59 |
| L* | 74 | 72 | 70 | 68 | 67 | 65 | 64 | 63 | 62 | 61 | 60 | 59 | 58 | 57 | 56 | 55 | 55 |
| a* | −15 | −10 | −5 | −2 | 2 | 5 | 8 | 10 | 13 | 15 | 18 | 20 | 22 | 24 | 26 | 28 | 30 |
| b* | 33 | 29 | 26 | 23 | 20 | 18 | 16 | 13 | 12 | 10 | 8 | 6 | 5 | 3 | 1 | 0 | −1 |

### 5. Determination of Masticatory Performance Using Color Scale

After masticating Masticatory Performance Evaluating gum XYLITOL a fixed number of times, it can be determined that the self performed mastication using a color scale corresponds to what number of times of mastication of the individuals with healthy teeth and jaw. By comparing the number of times of mastication obtained from the color scale of the individuals with healthy teeth and jaw with the number of times of actual mastication, the masticatory performance may be quantitatively evaluated as a ratio of the self masticatory performance to the masticatory performance of individuals with healthy teeth and jaw.

This application claims the benefit of Japanese Patent Application No. 2009-227154, filed Sep. 30, 2009, which is hereby incorporated by reference herein in its entirety.

1. A method for creating a color scale for determining a number of times of mastication of an individual with healthy teeth and jaw, using a gum with a color which can be changed depending on mastication, based on a color of the gum after mastication, the method comprising:
a first step of having a plurality of individuals with healthy teeth and jaw masticate the gum with a color changing depending on mastication for a predetermined number of times;
a second step of determining a regression equation representing a relationship between a coordinate value of a color of the gum after mastication and a color difference of the gum between before and after mastication in a color system;
a third step of determining a regression equation representing a relationship between the color difference and the number of times of mastication; and
a fourth step of determining an average number of times of mastication for the color of the gum after mastication from the regression equations obtained in the second and third steps.

2. The method for creating a color scale according to claim 1, wherein the gum with a color changing depending on mastication is Masticatory Performance Evaluating gum XYLITOL (trade name).

3. The method for creating a color scale according to claim 1, wherein the color system used is a CIE L*a*b* color system.

4. A color scale created by a method according to claim 1.

5. A method for determining masticatory performance of a subject, the method comprising:
a first step of determining a correlation between a coordinate value in a color system of a color of a gum with a color which can be changed depending on mastication after mastication and a number of times of mastication for an individual with healthy teeth and jaw; a second step of subsequently determining the number of times of mastication of the individual with healthy teeth and jaw, corresponding to the number of times of mastication of a subject, based on a color difference of gums before and after mastication; and a third step of comparing with the number of times of mastication of the subject.

6. The method for determining masticatory performance of a subject according to claim 5, wherein the number of times of mastication of an individual with healthy teeth and jaw is determined as zero in the case where a value of (L*, a*, b*) in a CIE L*a*b* color system after mastication of Masticatory Performance Evaluating gum XYLITOL is (74, −15, 33), the number of times of mastication is determined as 10 in the case of (72, −10, 29), the number of times of mastication is determined as 20 in the case of (70, −5, 26), the number of times of mastication is determined as 30 in the case of (68, −2, 23), the number of times of mastication is determined as 40 in the case of (67, 2, 20), the number of times of mastication is determined as 50 in the case of (65, 5, 18), the number of times of mastication is determined as 60 in the case of (64, 8, 16), the number of times of mastication is determined as 70 in the case of (63, 10, 13), the number of times of mastication is determined as 80 in the case of (62, 13, 12), the number of times of mastication is determined as 90 in the case of (61, 15, 10), the number of times of mastication is determined as 100 in the case of (60, 18, 8), the number of times of mastication is determined as 110 in the case of (59, 20, 6), the number of times of mastication is determined as 120 in the case of (58, 22, 5), the number of times of mastication is determined as 130 in the case of (57, 24, 3), the number of times of mastication is determined as 140 in the case of (56, 26, 1), the number of times of mastication is determined as 150 in the case of (55, 28, 0) and the number of times of mastication is determined as 160 in the case of (55, 30, −1).

7. The method for determining masticatory performance of a subject according to claim 5, wherein in the second step, a color scale is created based on the correlation obtained in the first step and the number of times of mastication in an individual with healthy teeth and jaw is determined using the color scale.
